(19) United States
(12) Patent Application Publication
(10) Pub. No.: US 2018/0369214 A1
(43) Pub. Date: Dec. 27, 2018

(54) Methods of Diagnosing and Treating Small Cell Lung Cancer Using Polo-Like Kinase 1 (PLK1) Inhibitors

(71) Applicant: Emory University, Atlanta, GA (US)

(72) Inventor: Taofeek Owonikoko, Atlanta, GA (US)

(21) Appl. No.: 16/020,291

(22) Filed: Jun. 27, 2018

Related U.S. Application Data
(60) Provisional application No. 62/525,467, filed on Jun. 27, 2017.

(51) Int. Cl. A61K 31/437; A61P 35/00

(52) U.S. Cl. CPC A61K 31/437; A61K 31/198; A61P 35/00

(57) ABSTRACT

This disclosure relates to methods of diagnosing and treating small cell lung cancer. In certain embodiments, this disclosure relates to methods of treating small cell lung cancer comprising administering an effective amount of a polo-like kinase 1 (PLK-1) inhibitor to a subject in need thereof. In certain embodiments, the subject is diagnosed with small cell lung cancer.

### CROSS-REFERENCE TO RELATED APPLICATIONS

This application claims the benefit of U.S. Provisional Application No. 62/525,467 filed Jun. 27, 2017. The entirety of this application is hereby incorporated by reference for all purposes.

### BACKGROUND

Small cell lung cancer (SCLC) is a significant health problem. The outcome for patients with SCLC remains bleak, and survival of newly diagnosed patients is typically estimated to be from weeks to months. As of 2015, not more than 5% will survive to 5 years. While a majority of SCLC patients respond to frontline chemotherapy, recurrence of disease is common sometimes within weeks of completing treatment. Recurrence following initial frontline therapy is associated with resistance to the available salvage treatment in the vast majority of cases. Thus, there is a need to identify improved therapies.

Wildey et al. report pharmacogenomic approach to identify drug sensitivity in small-cell lung cancer. PLoS One, 2014, 9(9): e106784.

Owonikoko et al. report patient-derived xenografts faithfully replicated clinical outcome in a phase II co-clinical trial of arsenic trioxide in relapsed small cell lung cancer. J Transl Med, 2016, 14: 111.

U.S. Pat. No. 8,648,078 reports inhibitors of PLK1 are useful in the treatment of proliferative disorders such as cancer. See also US. Published Patent Application No. 2014/0378338 and WO2012/058704.

References cited herein are not an admission of prior art.

### SUMMARY

This disclosure relates to methods of diagnosing and treating small cell lung cancer. In certain embodiments, this disclosure relates to methods of treating small cell lung cancer comprising administering an effective amount of a polo-like kinase 1 (PLK-1) inhibitor to a subject in need thereof. In certain embodiments, the subject is diagnosed with small cell lung cancer.

In certain embodiments, this disclosure relates to methods of treating small cell lung cancer comprising administering an effective amount of polo-like kinase 1 (PLK-1) inhibitor to a subject in need thereof, wherein the PLK inhibitor is selected from volasertib (BI-6727), rigosertib (ON-01910), R)-4-(8-cyclopentyl-7-ethyl-5-methyl-6-oxo-5,6,7,8-tetrahydropteridin-2-ylamino)-3-methoxy-N-(1-methylpiperidin-4-yl)benzamide (BI-2536), 5-(6-((4-methylpiperazin-1-yl)methyl)-1H-benzo[d]imidazol-1-yl)-3-((R)-1-(2-(trifluoromethyl)phenyl)ethoxy)thiophene-2-carboxamide (GSK461364), N-[(4-methoxyphenyl)sulfonyl]-N-[2-[(1E)-2-(1-oxido-4-pyridinyl)ethenyl]phenyl]-acetamide (HMN-214), 2-[[5-[3-(dimethylamino)propyl]-2-methyl-3-pyridinyl]amino]-5,7-dihydro-9-(trifluoromethyl)-6H-Pyrimido[5,4-d][1]benzazepine-6-thione (MLN0905), 4-[(9-cyclopentyl-7,7-difluoro-6,7,8,9-tetrahydro-5-methyl-6-oxo-5H-pyrimido[4,5-b][1,4]diazepin-2-yl)amino]-3-methoxy-N-(1-methyl-4-piperidinyl)-benzamide (Ro3280), 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075), 2-[3-[(1E)-2-[4-[[(2R,6S)-2,6-dimethyl-4-morpholinyl]methyl]phenyl]ethenyl]-1H-indazol-6-yl]-5′-methoxy-Spiro[cyclopropane-1,3′-[3H]indol]-2′(1′H)-one (CFI-400945), and 5-(5,6-Dimethoxy-1H-benzimidazol-1-yl)-3-[[2-(trifluoromethyl)phenyl] methoxy]-2-thiophenecarboxamide (GW843682X), or salts thereof.

In certain embodiments, administration of the polo-like kinase 1 (PLK-1) inhibitor is in combination with another chemotherapy agent. In certain embodiments, the subject is a human subject. In certain embodiments, this disclosure contemplates use as a first line treatment and use as a second line treatment, e.g., after growth of small cell lung cancer returns after a period of remission. In certain embodiments, the subject previously received a first chemotherapy treatment such as an administration schedule of etoposide, cisplatin, carboplatin, irinotecan, or combinations thereof.

In certain embodiments, this disclosure relates to methods of diagnosing and treating small cell lung cancer comprising, i) obtaining a sample from a subject; ii) identifying that the sample comprises a genetic alteration in gene TP53; iii) diagnosing the subject as responsive to a PLK inhibitor therapy when the sample is identified as comprising an alteration in gene TP53; iv) administering to the subject an effective amount of a PLK inhibitor. In certain embodiments, the subject is diagnoses with an inactivating, gain-of-function (GOF), or non-disruptive mutation in the in gene TP53. In certain embodiments, the sample is of a lung tissue, tumor, or cancer cell of the subject.

### BRIEF DESCRIPTION OF THE DRAWINGS

FIG. 1 shows data on in vitro cytotoxicity of targeted agents assessed in a panel of SCLC cell lines. PLK1 inhibitors, volasertib and rigosertib, showed potent and consistent activity against SCLC cell lines with picomolar IC₅₀ concentration against the cell lines.

FIG. 2 shows data on tumor growth inhibition by PLK1 inhibitor, volasertib, in xenograft tumor of H526 SCLC cell line.

FIG. 3 shows data tumor growth inhibition in a SCLC PDX model of platinum sensitive SCLC demonstrated significant tumor growth inhibition and reduced tumor weight by rigosertib compared to vehicle. Note the comparable efficacy by rigosertib and cisplatin, but no significant effect of arsenic compared to vehicle.

FIG. 4 shows data on shRNA knockdown of TP53 gene sensitized H292 and A549 NSCLC cell lines to PLK1 inhibitors, volasertib (BI-6727) and rigosertib (ON-01910) compared to parental and vector-treated controls.

FIG. 5 shows data indicating enforced expression of mutant P53 in a p53 null cell line (H1299) reduced sensitivity to PLK1 inhibitor.

FIG. 6 shows data from enforced expression of mutant p53 and depletion of wild type p53 in H1299 and H292 cell lines respectively. Note the increased PLK1 and CDC25 accumulation in the presence of P53 protein; P: Parent cell; V: Vector; H2: R273H mutant; H3: R175H mutant.

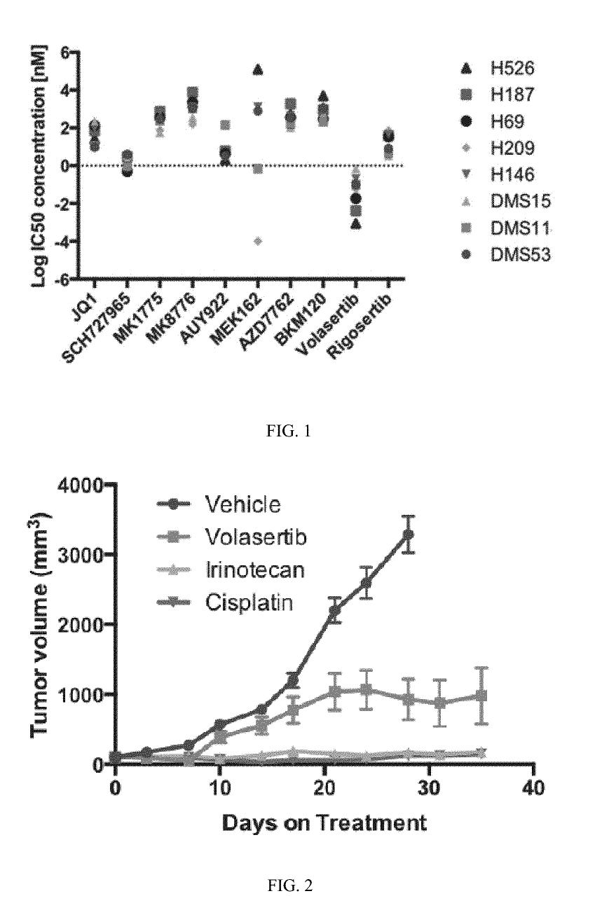

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Subject” refers to any animal, preferably a human patient, livestock, rodent, monkey or domestic pet.

A “chemotherapy agent,” “chemotherapeutic,” “anti-cancer agent” or the like, refer to molecules that are recognized to aid in the treatment of a cancer. Contemplated examples include the following molecules, prodrugs, or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed di sodium, copanli sib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelali sib, imatinib, talimogene laherparepvec, ipilimumab, romidepsin, ixabepilone, ixazomib, ruxolitinib, cabazitaxel, palifermin, pembrolizumab, ribociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the terms “small cell lung cancer” refers to small cell carcinoma (oat cell cancer) or combined small cell carcinoma identified in lung tissue. Tests and procedures may be used to detect (find), diagnose, and stage small cell lung cancer. A subject may be diagnosed with small cell lung cancer by laboratory tests, sputum cytology, lung biopsy, e.g., fine-needle aspiration (FNA) biopsy of the lung, bronchoscopy, thoracoscopy, thoracentesis, mediastinoscopy, CT scan (CAT scan), or chest x-ray. Test samples of lung tissue or fluid, blood, urine, or other substances in the body may be used. These tests may be used to plan and check treatment, or monitor the disease over time.

### Methods of Use

This disclosure relates to methods of diagnosing and treating small cell lung cancer. In certain embodiments, this disclosure relates to methods of treating small cell lung cancer comprising administering an effective amount of a polo-like kinase 1 (PLK-1) inhibitor to a subject in need thereof. In certain embodiments, the subject is diagnosed with small cell lung cancer.

In certain embodiments, this disclosure relates to methods of treating small cell lung cancer comprising administering an effective amount of polo-like kinase 1 (PLK-1) inhibitor to a subject in need thereof, wherein the PLK inhibitor is selected from volasertib (BI-6727), rigosertib (ON-01910), R)-4-(8-cyclopentyl-7-ethyl-5-methyl-6-oxo-5,6,7,8-tetrahydropteridin-2-ylamino)-3-methoxy-N-(1-methylpiperidin-4-yl)benzamide (BI-2536), 5-(6-((4-methylpiperazin-1-yl)methyl)-1H-benzo[d]imidazol-1-yl)-3-((R)-1-(2-(trifluoromethyl)phenyl)ethoxy)thiophene-2-carboxamide (GSK461364), N-[(4-methoxyphenyl)sulfonyl]-N-[2-[(1E)-2-(1-oxido-4-pyridinyl)ethenyl]phenyl]-acetamide (HMN-214), 2-[[5-[3-(dimethylamino)propyl]-2-methyl-3-pyridinyl]amino]-5,7-dihydro-9-(trifluoromethyl)-6H-Pyrimido[5,4-d][1]benzazepine-6-thione (MLN0905), 4-[(9-cyclopentyl-7,7-difluoro-6,7,8,9-tetrahydro-5-methyl-6-oxo-5H-pyrimido[4,5-b][1,4]diazepin-2-yl)amino]-3-methoxy-N-(1-methyl-4-piperidinyl)-benzamide (Ro3280), 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075), 2-[3-[(1E)-2-[4-[[(2R,6S)-2,6-dimethyl-4-morpholinyl]methyl]phenyl]ethenyl]-1H-indazol-6-yl]-5′-methoxy-Spiro[cyclopropane-1,3′-[3H]indol]-2′(1′H)-one (CFI-400945), and 5-(5,6-Dimethoxy-1H-benzimidazol-1-yl)-3-[[2-(trifluoromethyl)phenyl] methoxy]-2-thiophenecarboxamide (GW843682X), or salts thereof, or a lipid nanoparticle (LNP) encapsulated small interfering RNA (siRNA) directed against polo-like kinase 1 (PLK1).

In certain embodiments, administration of the polo-like kinase 1 (PLK-1) inhibitor is in combination with another chemotherapy agent. In certain embodiments, the subject is a human subject. In certain embodiments, this disclosure contemplates use as a first line treatment and use as a second line treatment after growth of small cell lung cancer returns after a period of remission. In certain embodiments, the subject previously received a first chemotherapy treatment such as an administration schedule of etoposide, cisplatin, carboplatin, irinotecan, or combinations thereof.

In certain embodiments, this disclosure relates to methods of treating small cell lung cancer comprising administering an effective amount of a polo-like kinase 1 (PLK-1) inhibitor in combination with cisplatin to a subject in need thereof.

In certain embodiments, this disclosure relates to methods of treating small cell lung cancer comprising administering an effective amount of a polo-like kinase 1 (PLK-1) inhibitor in combination with etoposide to a subject in need thereof.

In certain embodiments, this disclosure relates to methods of treating small cell lung cancer comprising administering an effective amount of a polo-like kinase 1 (PLK-1) inhibitor in combination with irinotecan to a subject in need thereof.

In certain embodiments, this disclosure relates to methods of treating small cell lung cancer comprising administering an effective amount of a polo-like kinase 1 (PLK-1) inhibitor in combination with carboplatin, to a subject in need thereof.

In certain embodiments, this disclosure relates to methods of treating small cell lung cancer comprising administering an effective amount of a polo-like kinase 1 (PLK-1) inhibitor in combination with cisplatin and etoposide to a subject in need thereof.

In certain embodiments, this disclosure relates to methods of treating small cell lung cancer comprising administering an effective amount of a polo-like kinase 1 (PLK-1) inhibitor in combination with carboplatin and etoposide to a subject in need thereof.

In certain embodiments, this disclosure relates to methods of treating small cell lung cancer comprising administering an effective amount of a polo-like kinase 1 (PLK-1) inhibitor in combination with carboplatin and irinotecan to a subject in need thereof.

In certain embodiments, this disclosure relates to methods of treating small cell lung cancer comprising administering an effective amount of rigosertib in combination with cisplatin to a subject in need thereof.

In certain embodiments, this disclosure relates to methods of treating small cell lung cancer comprising administering an effective amount of rigosertib in combination with etoposide to a subject in need thereof.

In certain embodiments, this disclosure relates to methods of treating small cell lung cancer comprising administering an effective amount of rigosertib in combination with irinotecan to a subject in need thereof.

In certain embodiments, this disclosure relates to methods of treating small cell lung cancer comprising administering an effective amount of rigosertib in combination with carboplatin, to a subject in need thereof.

In certain embodiments, this disclosure relates to methods of treating small cell lung cancer comprising administering an effective amount of rigosertib in combination with cisplatin and etoposide to a subject in need thereof.

In certain embodiments, this disclosure relates to methods of treating small cell lung cancer comprising administering an effective amount of rigosertib in combination with carboplatin and etoposide to a subject in need thereof.

In certain embodiments, this disclosure relates to methods of treating small cell lung cancer comprising administering an effective amount rigosertib in combination with carboplatin and irinotecan to a subject in need thereof.

In certain embodiments, this disclosure relates to methods of treating small cell lung cancer comprising administering an effective amount of volasertib in combination with cisplatin to a subject in need thereof.

In certain embodiments, this disclosure relates to methods of treating small cell lung cancer comprising administering an effective amount of volasertib in combination with etoposide to a subject in need thereof.

In certain embodiments, this disclosure relates to methods of treating small cell lung cancer comprising administering an effective amount of volasertib in combination with irinotecan to a subject in need thereof.

In certain embodiments, this disclosure relates to methods of treating small cell lung cancer comprising administering an effective amount of volasertib in combination with carboplatin, to a subject in need thereof.

In certain embodiments, this disclosure relates to methods of treating small cell lung cancer comprising administering an effective amount of volasertib in combination with cisplatin and etoposide to a subject in need thereof.

In certain embodiments, this disclosure relates to methods of treating small cell lung cancer comprising administering an effective amount of volasertib in combination with carboplatin and etoposide to a subject in need thereof.

In certain embodiments, this disclosure relates to methods of treating small cell lung cancer comprising administering an effective amount volasertib in combination with carboplatin and irinotecan to a subject in need thereof.

In certain embodiments, this disclosure relates to methods of treating small cell lung cancer comprising administering an effective amount of 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof in combination with cisplatin to a subject in need thereof.

In certain embodiments, this disclosure relates to methods of treating small cell lung cancer comprising administering an effective amount of 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof in combination with etoposide to a subject in need thereof.

In certain embodiments, this disclosure relates to methods of treating small cell lung cancer comprising administering an effective amount of 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof in combination with irinotecan to a subject in need thereof.

In certain embodiments, this disclosure relates to methods of treating small cell lung cancer comprising administering an effective amount of 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof in combination with carboplatin, to a subject in need thereof.

In certain embodiments, this disclosure relates to methods of treating small cell lung cancer comprising administering an effective amount of 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof in combination with cisplatin and etoposide to a subject in need thereof.

In certain embodiments, this disclosure relates to methods of treating small cell lung cancer comprising administering an effective amount of 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof in combination with carboplatin and etoposide to a subject in need thereof.

In certain embodiments, this disclosure relates to methods of treating small cell lung cancer comprising administering an effective amount 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof in combination with carboplatin and irinotecan to a subject in need thereof.

In certain embodiments, methods contemplate administration in cycles with a period of treatment of daily for 1 to 3 days followed by a rest period of at least one, two, three or more days. In certain embodiments, the cycle generally lasts about 2 to 4 weeks, and/or for 2 to 6 cycles, 2 to 7 cycles, or 2 to 8 cycles.

In certain embodiments, this disclosure contemplates methods wherein if during initial treatment cancer progresses during treatment or returns after treatment with etoposide, cisplatin, irinotecan, or combinations thereof, then the subject is administered a cycle of with polo-like kinase 1 (PLK-1) inhibitors disclosed herein optionally in combination with other chemotherapy agents.

In certain embodiments, this disclosure relates to methods of diagnosing and treating small cell lung cancer comprising, i) obtaining a sample from a subject; ii) identifying that the sample comprises a genetic alteration in gene TP53; iii) diagnosing the subject as responsive to a PLK inhibitor therapy when the sample is identified as comprising an alteration in gene TP53; iv) administering to the subject an effective amount of a PLK inhibitor. In certain embodiments, the subject is diagnoses with an inactivating, gain-of-function (GOF), or non-disruptive mutation in the in gene TP53. In certain embodiments, the sample is of tumor of the subject.

In certain embodiments, methods discloses herein may be used in combination with methods for removing or killing lung cancer cells, such as chemotherapy, radiation therapy, photodynamic therapy, laser therapy, stent placement, or surgery or the lungs.

In certain embodiments, methods discloses herein may be used in combination with procedures for alleviating fluid buildup in the area around the lungs and the heart such as thoracentesis, pleurodesis, catheter placement, pericardiocentesis, and creating a pericardial window.

### Using Polo-Like Kinase 1 (PLK1) Inhibitors in the Treatment of Small Cell Lung Cancer

While SCLC is genomically unstable and has one of the highest mutation burdens of all cancers, strategies to exploit these genetic alterations either as direct targets of therapy or as a guide to optimize clinical efficacy of anticancer therapy have not been systematically explored. Despite the abundant genetic alterations present in small cell lung cancer (SCLC), a personalized approach to guide patient treatment has not been identified.

A panel of SCLC cell lines was evaluated for an unbiased agnostic preclinical screening of biologically targeted agents as a way to uncover hitherto unrecognized promising therapeutic leads. This unprejudiced low throughput screening approach identified polo-like kinase 1 (PLK1) inhibitors as candidates for comprehensive preclinical evaluation and eventual clinical testing in SCLC patients. Rigosertib and volasertib are PLK1 inhibitors already in clinical development in hematologic malignancies. These compounds demonstrated significant in vitro cytotoxicity with low nanomolar to picomolar IC₅₀ concentrations against our panel of SCLC cell lines. Antitumor efficacy of these agents using traditional subcutaneous xenografts as well as patient derived xenograft (PDX) models of SCLC were confirmed. Furthermore, preliminary elucidation of putative biomarkers of efficacy revealed an association of specific subtypes of TP53 gene mutations with increased cytotoxicity.

PLK1 is oncogenic when ectopically expressed in cell lines, and naturally elevated expression is associated with aggressive tumor phenotype. P53 protein in coordination with the PLK1 promoter, CDE-CHR (cell-cycle dependent element and cell cycle gene homology region) serves as a key regulator that represses PLK1 gene transcription during G1. In addition, RB and the E2F family of transcription factors also function together to repress PLK1 gene expression. PLK1 is thus an integrative molecule whose catalytic activity is modulated through the normal function of P53 and RB protein. The near universal presence of genetic alterations in TP53 and RB1 genes in SCLC provides a rational biological explanation for the serendipitous finding of potent in vitro cytotoxic activity of PLK1 inhibitor against SCLC cell lines in preclinical screening.

The concurrent assessment of potential predictive biomarkers to improve the chances of success in later stages of clinical testing is an established approach that has not been previously incorporated into

SCLC clinical trials. Evaluating different types of TP53 genetic alterations, (i.e. inactivating, gain-of-function (GOF), non-disruptive mutations) as predictors of efficacy for PLK1 inhibitor therapy is away to conduct prospective clinical trials in SCLC.

### Examples

### Agnostic Cytotoxicity Screening of Targeted Agents in SCLC Cell Lines Identified PLK1 Inhibitors as Promising Therapeutic Agents for SCLC.

An agnostic screening approach was employed by evaluating in vitro cytotoxicity of biologically targeted agents that have not been previously evaluated in SCLC clinical trials. A priori, submicromolar in vitro cytotoxicity was established as the minimum threshold required in order for any of the targeted agents to be considered worthy of detailed preclinical testing and eventual evaluation in patients. The cytotoxicity of 10 different targeted agents was determined in a panel of genomically annotated SCLC cell lines by low throughput MTS assay. Inhibitors of the bromodomain, HSP90, CDK2, MEK, PIK3CA and PLK1 were tested in a panel of SCLC cell lines (DMS53, DMS114, DMS153, H146, H209, H69, H128, H187, H526) well characterized for genetic alterations and expression profiles. In vitro activity of agents targeting PLK1 (rigosertib, ON-01910, and volasertib, B16727); CDK2 (SCH727965); HSP90 (AUY922) and a bromodomain inhibitor (JQ-1) met the a priori threshold of activity. Picomolar sensitivity of a panel of SCLC cell lines to PLK inhibitors, Volasertib (BI6727) and Rigosertib (ON01910) was identified. Picomolar activity of PLK1 inhibitor (BI6727) showed no surviving viable cells at BI6727 concentration of 0.01 nM and higher in a representative SCLC cell line (H187).

Some promising compounds were deprioritized because of toxicity profile (SCH727965). Both rigosertib and volasertib demonstrated low nanomolar to picomolar activity in the tested SCLC cell lines. Moreover, the role of PLK1 as an integrative molecule that cross talk with both RB1 and P53 provides strong biological plausibility and relevance for inhibitors of this target as SCLC therapy.

### PLK1 Inhibitor, Volasertib, Showed In Vivo Efficacy Against Subcutaneous Xenograft Model of 11526 SCLC

To establish whether the promising in vitro activity of these PLK1 inhibitors translates into antitumor efficacy in vivo. The activity of PLK1 inhibitors were compared to that of approved drugs currently employed in the management of SCLC. The in vivo efficacy of volasertib (30 mg/kg i.p. weekly), cisplatin (3 mg/kg i.p. weekly) and irinotecan (25 mg/kg i.p. weekly) was tested, using a subcutaneous xenograft model of the H526 cell line, the most sensitive cell line from our in vitro screen (FIG. 2). Tumor xenografts were established in 6-week old athymic (nu/nu) mice using H526 cells (1-2×10⁷) suspended in serum-free medium mixed with Matrigel™ solution and injected subcutaneously into the flank region of nude mice. Treatment began in groups of tumor-bearing mice (approximately 6 mice per group) matched for body weight and tumor volume when the tumors achieved a volume of approximately 100 mm³. Volasertib achieved significant tumor growth inhibition (p<0.003) relative to control in this subcutaneous xenograft model of H526 SCLC cell line (FIG. 2).

### PLK1 Inhibitor, Rigosertib, Showed In Vivo Efficacy Against Patient Derived Xenograft (PDX) Model of SCLC

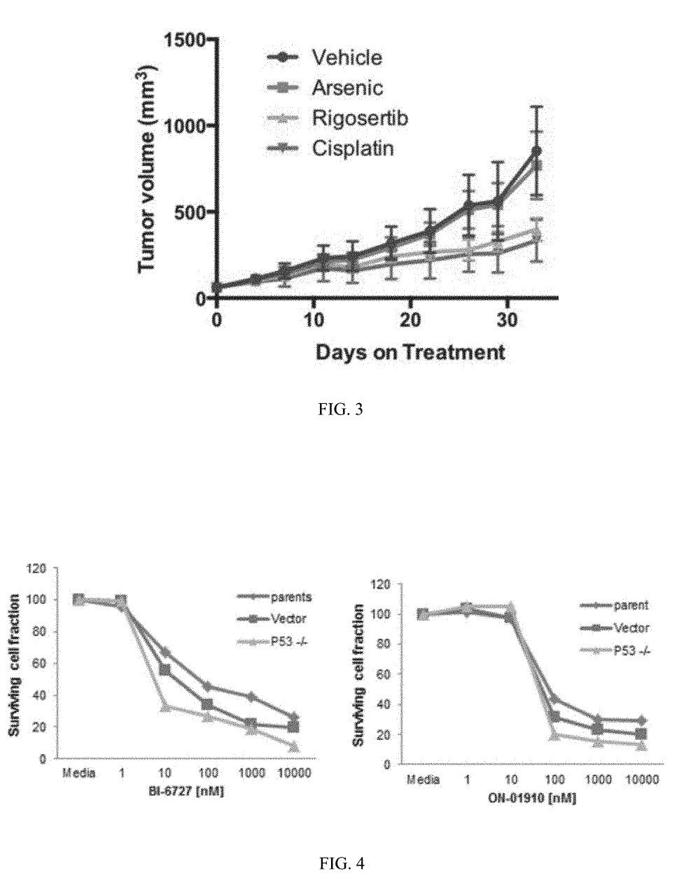

PDX models generated from SCLC patients enrolled in a recently completed translational phase II clinical trial of arsenic trioxide were employed. TKO-005 is a PDX representative of platinum sensitive SCLC from our tumor bank to test the efficacy of another PLK1 inhibitor, rigosertib (ON01910, Na), which also showed potent in vitro cytotoxicity against SCLC cell lines in the preclinical screening work (FIG. 2). The activity of rigosertib was compared to that of cisplatin (expected to be active in a PDX from a platinum sensitive tumor) and arsenic trioxide (expected to have no efficacy based on the clinical trial outcome for the patient whose tumor biopsy was used to generate the PDX). Rigosertib achieved significant tumor growth inhibition comparable to cisplatin in this PDX model of SCLC (FIG. 3) further confirming the promising activity of PLK1 inhibitor in SCLC. Arsenic trioxide was no better than vehicle further confirming the predictive power of the PDX model.

Expression of c-Myc but not PLK1 is Associated with PLK1 Inhibitor Cytotoxicity in SCLC Cell Lines

Both volasertib and rigosertib showed picomolar to nanomolar range cytotoxicity in SCLC cell lines. However, there were log range differences in the IC₅₀ concentrations between the cell lines, suggesting a differential sensitivity to PLK1 inhibitor that may have clinical impact. Unique genetic differences between the more sensitive and the less sensitive SCLC cell lines were elucidated (mean IC₅₀ concentration 0.03 nM vs. 0.25M; p=0.0113; Table 1).

TABLE 1
Cell lines sensitivity to volasertib and TP53 gene status

| Cell Line | IC50 (nM) | TP53 status |
|---|---|---|
| DMS53 | 0.118 | c.722C > T, |
| DMS153 | 0.672 | c.463A > C |
| H146 | 0.163 | Wild type |
| H209 | 0.057 | c.673-2A > T |
| H69* | 0.018 | c.511G > T |
| H187* | 0.004 | c.722C > G |
| H526* | 0.0008 | c.97-1G > C |
| DMS114* | 0.092 | c.637C > T |

*indicates concurrent hemizygous deletion

Native gene expression profiling data established for a panel of SCLC cell was interrogated, deposited in the NCBI Gene Expression Omnibus under the GEO accession number GSE55830. Total RNA was isolated using RNeasy™ while total RNA sample quality and concentrations were determined using NanoDrop™ and Agilent 2100 Bioanalyzer™. Each sample was prepared for Illumina Human HT-12 v4 Expression BeadChips™ according to the manufacturer's protocol. The HT-12 platform contains over 47000 probes that cover well-characterized genes, gene candidates, and splice variants. BeadChips™ were scanned on the Illumina HiScan™ instrument to determine probe fluorescence intensity. The raw probe intensities were normalized using the quantile normalization algorithm. The log-2 transformed expression data was correlated with the degree of sensitivity to PLK1 inhibitor by comparing the normalized gene expression for specific genes of interest between the more sensitive (Low IC₅₀) to the less sensitive (High IC₅₀) cell lines using the median IC₅₀ concentration for the entire panel of cell lines as the cut-point. The expression of c-Myc was significantly lower in the most sensitive cell lines with a similar but less pronounced trend for PLK1; TP53 and RB1 expression was comparable between both groups.

TP53 Gene Mutation is Associated with PLK1 Inhibitor Cytotoxicity in Lung Cancer Cell Lines

Some studies report cells with a normal diploid karyotype are insensitive to PLK1 depletion indicating cancer cell selectivity and reduced potential for off-target toxicity. However, another report suggested no significant association between TP53 mutation and cancer cell vulnerability to PLK1 inhibition. Perhaps, failure to account for different types of TP53 mutations, which may have opposite impact on p53 protein function could account for this discordance. To assess whether differences exist between specific subtypes of TP53 gene mutation and sensitivity to PLK1 inhibition, the TP53 genetic alterations were characterized in the cell lines using SNaPshot™ a targeted DNA sequencing platform. Mutations in all the coding exons of the TP53 gene were assesses. There was a high prevalence of TP53 gene mutation in the cell lines (7 of 8 cell lines). Intriguingly, the most sensitive cell line, H526 harbors an inactivating TP53 mutation in one allele and hemizygous deletion of the other allele. Three other cell lines with concomitant deletion of the second allele of TP53 were also much more sensitive than cell lines with wild type or without concomitant hemizygous deletion (Table 1).

shRNA Inactivation of TP53 Sensitizes Cancer Cells to PLK1 Inhibition while Enforced Expression of Gain-of-Function Mutant Confers Resistance to PLK1

Whether depletion of endogenous wild type P53 and enforced expression of active P53 mutant would alter cancer cell line sensitivity to PLK1 inhibitor was evaluated. Due to low transfection efficiency in SCLC cell lines, established NSCLC cell line models were relied on to interrogate PLK1 activity following TP53 gene modulation. In vitro cytotoxicity of PLK1 inhibitors were compared before and after shRNA knockdown of TP53 in H292 and A549. In these cell lines with wild type TP53, there was increased sensitivity to PLK1 inhibitors following shRNA knockdown in comparison to parent cells and vector treated controls (FIG. 4). We also evaluated PLK1 inhibitor activity in p53−/− H1299 cells following enforced expression of active mutant forms of TP53. There was reduced sensitivity of the mutant-bearing cell lines compared to parental p53−/− cells (FIG. 5). Overall, these results suggest that total loss or inactivation of TP53 confers vulnerability to PLK1 inhibitor therapy in cancer cells while a GOF mutation is more likely to reduce sensitivity to PLK1 inhibitor.

### Heterogeneity of TP53 Mutations in SCLC

TP53 is frequently mutated genes in human cancers. However, the frequency of TP53 alterations is highly variable from one type of cancer to another, ranging from less than 5% in cervical carcinoma to 80-90% in SCLC and 90% in ovarian carcinoma. TP53 is a direct transcription activator for hundreds of genes but can also act as a transcription repressor. Overall, non-synonymous single-nucleotide variants are the most common deleterious genetic alterations of TP53 but other mechanisms such as gene deletion are reported in some tumor types such as osteosarcoma. Furthermore, inactivation of genes that closely interact with the TP53 networks (e.g. MDM2, MDMX and p19ARF) could also confer the phenotype of TP53 inactivation.

There are about 45,000 distinct mutations reported in TP53 and catalogued in various databases. Of these, 1,540 variants are single-nucleotide substitutions while 2,000 represent frameshift mutants arising as a result of small insertions or deletions. The specific location of the mutation event in a hot spot in exons 5-8 encoding the DNA binding domain of the TP53 protein is important to P53 function. Disruptive mutations are defined as non-conservative mutations located inside the key DNA-binding domain (L2 (codons 163-195)-L3 region (236-251)), or stop codons in any region, while the non-disruptive mutations are conservative mutations or non-conservative mutations outside the L2-L3 region excluding stop codons. While the majority of these mutations are believed to be drivers of oncogenesis, a significant number likely represent passenger, non-driver mutations. Published databases and algorithms (e.g. TP53 database; Sift, Mut Assessor) are currently available for classifying TP53 gene alterations as activating, inactivating, sequencing artifacts or passenger alterations. TP53 gene mutation as a foundational genetic alteration in SCLC is nearly universal in all cases. However, the spectrum of biological effect may vary between depending on the specific type of genetic alteration and the involved gene loci. Publically available databases were employed to characterize the different types of TP53 mutations in 50 SCLC cell lines (Cancer Cell Line Encyclopedia) and 166 tissue samples

(TCGA). The majority of SCLC cell lines and tissue samples harbored TP53 mutation. However, there was significant variability with respect to the type and location of these alterations on the TP53 gene. Approximately, 60% of the alterations were disruptive and likely to significantly impact TP53 function while the remaining 40% were non-disruptive alterations predicted to not significantly impact p53 function. Differences in the biological consequence of disruptive inactivating TP53 mutations versus non-disruptive or GOF alterations in SCLC can be exploited to individualize therapy. For instance, the negative interaction between TP53 and PLK1 genes and our preliminary data suggest that inactivating, disruptive TP53 mutations rather than GOF or non-disruptive mutations will confer sensitivity to PLK1 inhibitors.

### Inactivating TP53 Gene Mutations Confer Vulnerability to PLK1 Inhibitors in SCLC

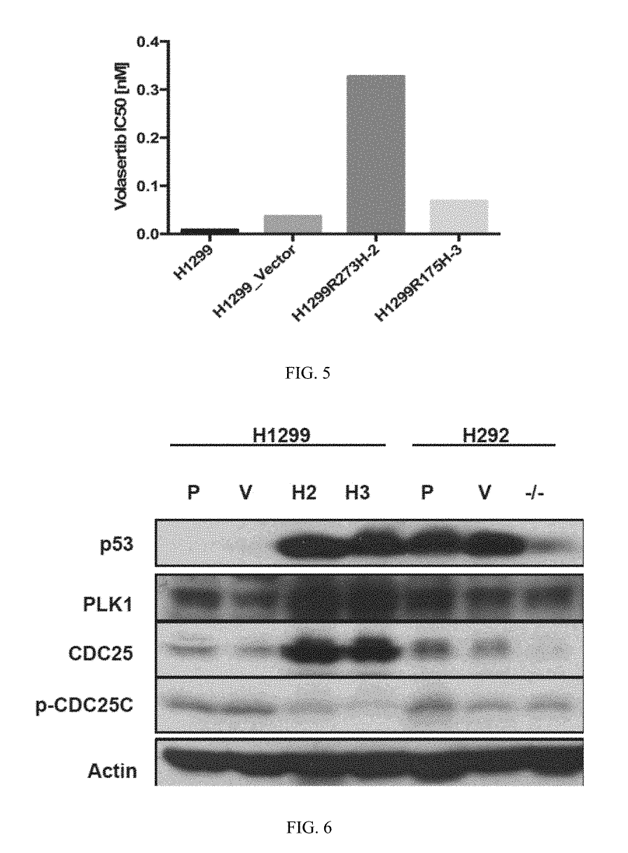

PLK1 is implicated in carcinogenesis of various human tumors and is the target of promising anticancer agents such as rigosertib and volasertib. Agnostic screening of targeted anticancer agents in SCLC identified PLK1 as a lead candidate for therapeutic targeting in SCLC. Specifically, two PLK1 inhibitors, volasertib and rigosertib, demonstrated potent in vitro cytotoxicity and in vivo efficacy in a panel of well-characterized SCLC cell lines and representative traditional xenograft and PDX models of SCLC. There was an association between alterations in TP53 gene and low c-Myc gene expression with cell line sensitivity to PLK1 inhibitor. Furthermore, depletion of wt p53 in H292 cell line and enforced expression of mutant p53 protein in H1299 cell line reproducibly modulated PLK1 and its downstream substrate CDC25 (FIG. 6). While TP53 and c-Myc genes do not currently present direct therapeutic targets, their central role in the maintenance of genomic fidelity and as drivers of tumor growth and resistance to therapy can be exploited to guide the development of targeted agents in this disease.

1. A method of treating small cell lung cancer comprising administering an effective amount of rigosertib to a subject in need thereof.

2. The method of claim 1, wherein the subject is diagnosed with small cell lung cancer.

3. The method of claim 1, wherein the subject previously received a first chemotherapy treatment.

4. The method of claim 3, wherein the first chemotherapy treatment was administering etoposide, cisplatin, carboplatin, irinotecan, or combinations thereof.

5. The method of claim 1, wherein administration of rigosertib is in combination with another chemotherapy agent.

6. The method of claim 1, wherein the subject is a human subject.

7. A method of treating small cell lung cancer comprising administering an effective amount of a compound 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salts thereof to a subject in need thereof.

8. The method of claim 7, wherein the subject is diagnosed with small cell lung cancer.

9. The method of claim 7, wherein the subject previously received a first chemotherapy treatment.

10. The method of claim 9, wherein the first chemotherapy treatment was administering etoposide, cisplatin, irinotecan, or combinations thereof.

11. The method of claim 7, wherein administration of 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075) or salt thereof is in combination with another chemotherapy agent.

12. The method of claim 7, wherein the subject is a human subject.

13. A method of diagnosing and treating small cell lung cancer comprising,
i) obtaining a sample from a subject;
ii) identifying that the sample comprises genetic alterations gene TP53;
iii) diagnosing the subject as responsive to a PLK inhibitor therapy when the sample is identified as comprising an alteration in gene TP53;
iv) administering to the subject administering an effective amount of a PLK inhibitor to the subject.

14. The method of claim 13, wherein the subject is diagnoses with an inactivating, gain-of-function (GOF), or non-disruptive mutation in the in gene TP53.

15. The method of claim 13, wherein the sample is of tumor of the subject.

16. The method of claim 13, wherein the PLK inhibitor is selected from volasertib (BI-6727), rigosertib (ON-01910), R)-4-(8-cyclopentyl-7-ethyl-5-methyl-6-oxo-5,6,7,8-tetrahydropteridin-2-ylamino)-3-methoxy-N-(1-methylpiperidin-4-yl)benzamide (BI-2536), 5-(6-((4-methylpiperazin-1-yl)methyl)-1H-benzo[d]imidazol-1-yl)-3-((R)-1-(2-(trifluoromethyl)phenyl)ethoxy)thiophene-2-carboxamide (GSK461364), N-[(4-methoxyphenyl)sulfonyl]-N-[2-[(1E)-2-(1-oxido-4-pyridinyl)ethenyl]phenyl]-acetamide (HMN-214), 2-[[5-[3-(dimethylamino)propyl]-2-methyl-3-pyridinyl]amino]-5,7-dihydro-9-(trifluoromethyl)-6H-Pyrimido[5,4-d][1]benzazepine-6-thione (MLN0905), 4-[(9-cyclopentyl-7,7-difluoro-6,7,8,9-tetrahydro-5-methyl-6-oxo-5H-pyrimido[4,5-b][1,4]diazepin-2-yl)amino]-3-methoxy-N-(1-methyl-4-piperidinyl)-benzamide (Ro3280), 4,5-dihydro-1-(2-hydroxyethyl)-8-[[5-(4-methyl-1-piperazinyl)-2-(trifluoromethoxy)phenyl]amino]-1H-pyrazolo[4,3-h]quinazoline-3-carboxamide (PMC-075), 2-[3-[(1E)-2-[4-[[(2R,6S)-2,6-dimethyl-4-morpholinyl]methyl]phenyl]ethenyl]-1H-indazol-6-yl]-5′-methoxy-Spiro[cyclopropane-1,3′-[3H]indol]-2′(1′H)-one (CFI-400945), and 5-5,6-Dimethoxy-1H-benzimidazol-1-yl)-3-[[2-(trifluoromethyl)phenyl] methoxy]-2-thiophenecarboxamide (GW843682X).

17. The method of claim 13, wherein the subject previously received a first chemotherapy treatment.

18. The method of claim 17, wherein the first chemotherapy treatment was etoposide, cisplatin, carboplatin, irinotecan, or combinations thereof.

19. The method of claim 13, wherein administration of the PLK inhibitor is in combination with another chemotherapy agent.

20. The method of claim 13, wherein the subject is a human subject.
